(19) United States
(12) Patent Application Publication
(10) Pub. No.: US 2002/0164299 A1
(43) Pub. Date: Nov. 7, 2002

(54) TNF and IFN stimulated genes and uses therefor

(76) Inventor: Grace Wong, Brookline, MA (US)

Correspondence Address: Dike, Bronstein, Roberts & Cushman, Intellectual Property Patent Practice, EDWARDS & ANGELL, LLP, 130 Water Street, Boston, MA, 02109, US

(21) Appl. No.: 09/854,432

(22) Filed: May 11, 2001

Related U.S. Application Data
(60) Provisional application No. 60/203,624, filed on May 12, 2000.

(51) Int. Cl. G01N 33/53; G01N 33/567; A61K 38/19

(52) U.S. Cl. 424/85.1; 435/7.2

(57) ABSTRACT

The present invention is directed to the identification of genes that are expressed at a higher level in certain TNF & IFN treated cells than in otherwise identical untreated cells. Genes that are expressed at a higher level in TNF & IFN treated cells than untreated cells (“TNF & IFN stimulated genes”) are of interest, in part, because TNF & IFN can or could influence a wide range of cellular processes and responses for antiviral activity. The identified TNF & IFN stimulated genes and the proteins they encode can be used: 1) as therapeutic agents which modulate a cellular process or response that is influenced by TNF & IFN; 2) as targets for use in high throughput screening and the development of therapeutic agents which modulate a cellular process or response that is influenced by TNF & IFN; and 3) as markers which can be used to detect and monitor a cellular process or response that is influenced by TNF & IFN.

This application claims the benefit of U.S. Provisional application No. 60/203,624, filed on May 12, 2001.

### BACKGROUND OF THE INVENTION

Interferons (IFNs) have been used to varying effect for the prophylaxis or treatment of chronic, acute and/or experimental infections by viruses such as vaccinia, rubella, herpes simplex, varicella-zoster, chicken pox, cytomegalovirus, adenovirus, ebola virus, rabies and hepatitis B. Tumor Necrosis Factor (TNF), both α and β forms, were initially described as tumoricidal proteins that are produced by activated macrophages and lymphocytes. Both TNF-α and TNF-β have antiviral activity and synergize with INFs in the induction of resistance to both RNA and DNA virus infection in diverse cell types. Accordingly, there is considerable interest in the identification of agents which can modulate cellular processes or responses influenced the synergism for antiviral activity of IFNs and TNFs, and markers which can be used to monitor cellular processes or responses influenced by the synergism for antiviral activity of IFNs and TNFs.

### SUMMARY OF THE INVENTION

The present invention is directed to the identification of genes that are expressed at a higher level in certain TNF & IFN treated cells than in otherwise identical untreated cells. Genes that are expressed at a higher level in TNF & IFN treated cells than untreated cells (“TNF & IFN stimulated genes”) are of interest, in part, because TNF & IFN can or could influence a wide range of cellular processes and responses for antiviral activity. The identified TNF & IFN stimulated genes and the proteins they encode can be used: 1) as therapeutic agents which modulate a cellular process or response that is influenced by TNF & IFN; 2) as targets for use in high throughput screening and the development of therapeutic agents which modulate a cellular process or response that is influenced by TNF & IFN; and 3) as markers which can be used to detect and monitor a cellular process or response that is influenced by TNF & IFN.

The TNF & IFN stimulated genes of the invention were identified using a nucleic acid microarray available from Incyte, Inc. and was used to determine which of approximately 8000 pre-selected nucleic acid sequences (genes) are more highly expressed in TNF & IFN treated Y1 cells.

Thus, the invention features a number of “TNF & IFN stimulated genes.” These are genes which are expressed at a relatively high level in TNF & IFN treated Y1 cells and which are not expressed (or are expressed at a relatively low level) in otherwise identical untreated cells. These genes are listed in Tables 1-3.

In one embodiment, the invention provides genes and gene products which can be used to modulate a cellular response or process which is influenced by TNF & IFN.

The present invention further provides genes and gene products which can be used to screen for or design agents which can be used to modulate a cellular response or process which is influenced by TNF & IFN. Thus, the genes of the present invention (Tables 1 and 2) can be used as in the development of treatments (either single agent or multiple agent) for treatment of diseases and disorders involving viral activity. For example, if increased expression of a selected TNF & IFN stimulated gene triggers an unwanted response, the gene or the protein encoded by the gene can be used to screen for therapeutic agents which decrease expression or activity of the protein encoded by the selected gene. For example, the expression of the selected TNF & IFN stimulated gene by an TNF & IFN treated cell can be measured in the presence and absence of a various test agents (compounds), permitting the identification of those agents which increase or decrease expression of the selected gene.

The invention also provides markers which can be used to detect or monitor a cellular response or process that is influenced by TNF & IFN. Thus, the markers can be used to diagnose disorders associated with a TNF & IFN influenced cellular response or process. The markers can also be used to determine whether a selected patient suffering from a disorder associated with an TNF & IFN influenced cellular response or process is likely to benefit from a therapy which alters the activity or expression of an TNF & IFN stimulated gene. For example, if a given disorder is caused by increased expression of a particular TNF & IFN stimulated gene, it may be possible to treat the disorder in patients having increased expression of the TNF & IFN stimulated gene by decreasing expression of the TNF & IFN stimulated gene.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the detailed description and from the claims. Although materials and methods similar or equivalent to those described herein can be used in the practice or testing of the invention, the preferred materials and methods are described below.

### DETAILED DESCRIPTION OF THE INVENTION

The present invention is based, in part, on the identification of genes whose expression in Y1 cells is increased by treatment with TNF & IFN (“TNF & IFN stimulated genes”). The TNF & IFN stimulated genes of the present invention as summarized in Tables 1 and 2. Each Table is based on the Incyte, Inc. mouse cDNA array.

The increased expression of a given TNF & IFN stimulated gene can be directly involved in or responsible for an TNF & IFN influenced cellular response or process such that modulation of the expression or activity of the gene product will modulate the TNF & IFN influenced cellular process or response. Such genes are generally referred to as target genes. Such genes and the products they encode can be used to modulate a combined TNF & IFN influenced cellular response or process. They may also be used to develop therapeutic agents which decrease or increase the expression or activity of the protein encoded by the selected gene. Thus, the expression of the selected gene in a TNF & IFN treated cell can be measured in the presence and absence of a various test agents (compounds), permitting the identification of those agents which modulate expression of the selected gene. Similarly, the activity of the product of the selected gene in an TNF & IFN treated cell can be measured in the presence and absence of a various test agents (compounds), permitting the identification of those agents which modulate the activity of the product of the selected gene

The increased expression of a given TNF-& IFN stimulated gene can be associated with or correlated with a given TNF & IFN influenced cellular response or process, but not be directly involved in the TNF & IFN influence process or response. Modulation of the expression or activity of the protein encoded by such a TNF & IFN stimulated gene will generally not modulate the TNF & IFN influenced cellular process or response. Such genes and their products useful as markers which can be used to detect or monitor a cellular process or response that is influenced by TNF & IFN. Of course, target genes and their products are similarly useful as markers which can be used to detect or monitor a cellular process or response that is influenced by TNF & IFN.

Accordingly, the present invention provides methods for modulating an TNF & IFN influenced cellular process or response in a patient by administering a TNF & IFN stimulated gene (Tables 1 and 2) or the product thereof.

The present invention also provides a method for identifying an agent which modulates an TNF & IFN influenced cellular process or response, the method comprising:

a) exposing a sample of cells to TNF & INF;

b) determining the level of expression in the sample of cells of one or more INF & IFN stimulated genes (Table 1 and 2) in the presence and absence of a selected agent; and

c) identifying that the agent modulates an TNF & IFN influenced cellular process or response when the expression of the one or more TNF & IFN stimulated genes in the cell sample in the presence of the agent differs from the expression of the one or more TNF & IFN stimulated genes in the absence of the agent.

The invention also provides a method for identifying an agent which modulates an TNF & IFN influenced cellular process or response, the method comprising:

a) exposing a sample of cells to TNF & IFN;

b) determining the activity in the sample of cells of the product of one or more TNF & IFN stimulated genes (Table 1 and 2) in the presence and absence of a selected agent; and identifying that the agent modulates an TNF & IFN influenced cellular process or response when the activity of the product of the one or more TNF & IFN stimulated genes in the cell sample in the presence of the agent differs from the activity of the product of the one or more TNF & IFN stimulated genes in the absence of the agent.

Agents which modulate an TNF & IFN influenced cellular process can also be identified using methods which entail assessing the effect of the agent on expression or activity of TNF & IFN stimulated genes or gene products in the absence of TNF & IFN.

Thus, invention provides a method for identifying an agent which modulates an TNF & IFN influenced cellular process or response, the method comprising:

a) providing a sample of cells;

b) determining the level of expression in the sample of cells of one or more TNF & IFN stimulated genes (Table 1 and 2) in the presence and absence of a selected agent; and

c) identifying that the agent modulates an TNF & IFN influenced cellular process or response when the expression of the one or more TNF & IFN stimulated genes in the cell sample in the

d) esence of the agent differs from the expression of the one or more TNF & IFN stimulated genes in the absence of the agent.

The invention also provides a method for identifying an agent which modulates an TNF & IFN influenced cellular process or response, the method comprising:

a) providing a sample of cells;

b) determining the activity in the sample of cells of the product of one or more TNF & IFN stimulated genes (Tables 1 and 2) in the presence and absence of a selected agent; and

c) identifying that the agent modulates an TNF & IFN influenced cellular process or response when the activity of the product of the one or more TNF & IFN stimulated genes in the cell sample in the presence of the agent differs from the activity of the product of the one or more TNF & IFN stimulated genes in the absence of the agent.

In all of the above-described methods for identifying an agent which modulates an TNF & IFN influenced cellular process or response, the preferred TNF & IFN stimulated genes are those which are target genes.

The invention also provides a method for detecting or monitoring a cellular process or response that is influenced by TNF & IFN, the method comprising:

a) obtaining a sample of cells from a patient;

b) determining the level of expression in the sample of cells of one or more TNF & IFN stimulated genes (Tables 1 and 2); and

c) identifying that the cells in the sample of cells obtained from the patient are undergoing a cellular process or response that is influenced by TNF & IFN when the level of expression of the one or more TNF & IFN stimulated genes in the cell sample is increased relative to the activity of the one or more TNF & IFN stimulated genes in a control the sample.

The invention also provides a method for detecting or monitoring a cellular process or response that is influenced by TNF & IFN, the method comprising:

a) obtaining a sample of cells from a patient;

b) determining the level of activity in the sample of cells of the product of one or more TNF & IFN stimulated genes (Tables 1 and 2); and

c) identifying that the cells in the sample of cells obtained from the patient are undergoing a cellular process or response that is influenced by TNF & IFN when the level of activity of the product of the one or more TNF & IFN stimulated genes in the cell sample is increased relative to the activity of the product of the one or more TNF & IFN stimulated genes in the control sample.

### Measurement of Expression in Diagnostic or Screening Methods

As used herein, the level or amount of expression of a gene refers to the absolute level of expression of an mRNA encoded by the gene or the absolute level of expression of the protein encoded by the gene.

Often, it is preferable to determine the expression of two or more of the identified genes, more preferably, three or more of the identified genes. Thus, it is preferable to assess the expression of a panel of genes.

As an alternative to making determinations based on the absolute expression level of selected gene(s), e.g, one or more TNF & IFN stimulated genes selected from the genes of Tables 1 and 2 determinations may be based on the normalized expression levels. Expression levels are normalized by correcting the absolute expression level of an TNF & IFN stimulated gene or a by comparing its expression to the expression of a gene that is not an TNF & IFN stimulated gene, e.g., a housekeeping genes that is constitutively expressed. Suitable genes for normalization include housekeeping genes such as the actin gene. This normalization allows one to compare the expression level in one sample, e.g., a patient sample, to another sample, e.g., a patient sample collected at an earlier time, or between samples from different sources.

Alternatively, the expression level can be provided as a relative expression level. To determine a relative expression level of a gene, the level of expression of the gene is determined for 10 or more samples, preferably 50 or more samples, prior to the determination of the expression level for the sample in question. The mean expression level of each of the genes assayed in the larger number of samples is determined and this is used as a baseline expression level for the gene(s) in question. The expression level of the gene determined for the test sample (absolute level of expression) is then divided by the mean expression value obtained for that gene.

Preferably, the samples used will be from similar tissues. The choice of the cell source is dependent on the use of the relative expression level data. For example, in order to determine whether a particular tissue will be relatively affected, using tissues of similar types for obtaining a mean expression score is preferred.

Using expression found in normal cells or cells which are not exposed to TNF & IFN as a mean expression score aids in validating whether the gene assayed is specific for an TNF & IFN influenced cellular process or response. Such a later use is particularly important in identifying whether a given TNF & IFN stimulated gene can serve as a target gene. In addition, as more data is accumulated, the mean expression value can be revised, providing improved relative expression values based on accumulated data.

The expression level can be measured in a number of ways, including, but not limited to: measuring the mRNA encoded by the selected genes; measuring the amount of protein encoded by the selected genes; or measuring the activity of the protein encoded by the selected genes.

The mRNA level can be determine in in situ and in in vitro formats using methods known in the art. Many of such methods use isolated RNA. For in vitro methods, any RNA isolation technique that does not select against the isolation of mRNA can be utilized for the purification of RNA from cells (see, e.g., Ausubel et al., eds., 1987-1997, Current Protocols in Molecular Biology, John Wiley & Sons, Inc. New York). Additionally, large numbers of tissue samples can readily be processed using techniques well known to those of skill in the art, such as, for example, the single-step RNA isolation process of Chomczynski (I 989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic methods for the detection of mRNA levels involves contacting the isolated mRNA with a nucleic acid molecule (probe) that can hybridize to the mRNA encoded by the gene being detected. In one format, the mRNA is immobilized on a solid surface and contacted with the probes, for example by running the isolated mRNA on an agarose gel and transferring the mRNA from the gel to a membrane, such a nitrocellulose. In an alternative format, the probes are immobilized on a solid surface and the mRNA is contacted with the probes, for example in a gene microarray of the type available from Incyte, Inc. A skilled artisan can readily adapt known mRNA detection methods for use in detecting the level of mRNA encoded by one or more of the identified TNF & IFN stimulated genes. An alternative method for determining the level of mRNA in a sample that is encoded by one of the genes of the present invention involves the process of nucleic acid amplification, e.g., by rtPCR (the experimental embodiment set forth in Mullis, 1987, from about 50 to 200 nucleotides in length). Under appropriate conditions with appropriate reagents, amplification primers result in the production of nucleic acid molecule comprising the nucleotide sequence flanked by the primers. A skilled artisan can readily determine appropriate primers (both nucleotide sequence and length) for amplifying and detecting the TNF & IFN stimulated genes of the present invention using art known methods and the nucleotide sequence of the TNF & IFN stimulated genes of the present invention.

A variety of methods can be used to determine the level of protein encoded by one or more of the TNF & IFN stimulated genes of the present invention. In general, these methods involve the use of a compound that selectively binds to the protein, for example an antibody.

Proteins can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to enzyme immunoassay (EIA), radioimmunoassay (RIA), Western blot analysis and enzyme linked immunoabsorbant assay (ELISA). A skilled artisan can readily adapt know protein/antibody detection methods for use in determining whether a cell expresses a protein encoded by one or more of the genes of the present invention.

In one format, antibodies, or antibody fragments, can be used in methods such as Western blots or immunofluorescence techniques to detect the expressed proteins. In such uses, it is generally preferable to immobilize either the antibody or protein on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, proteins isolated from cells can be run on a polyacrylamide gel electrophoresis and immobilized onto a solid phase support such as nitrocellulose. The support can then be washed with suitable buffers followed by treatment with the delectably labeled antibody that selectively binds a protein encoded by an TNF & IFN stimulated gene of the invention. The solid phase support can then be washed with the buffer a second time to remove unbound antibody. The amount of bound label on the solid support can then be detected by conventional means.

Another embodiment of the present invention includes a step of detecting whether an agent alters the expression of one or more of the TNF & IFN stimulated genes of the present invention. Although the present TNF & IFN stimulated genes were identified as being expressed in cells that were not being exposed to a potential therapeutic agent, treatment with an agent may, or may not, alter expression. Such alterations in the expression level of these genes can provide a further indication as to whether the cells will be responsive to treatment with the agent. In such a use, the present invention provides methods for assessing whether the cells will be responsive to an agent which modulates an TNF & IFN influenced cellular process or response, the method comprising:

a) exposing a sample of cells obtained from a patient to a test agent;

b) determining the level of expression of one or more genes TNF & IFN stimulated genes (Tables 1 and 2) in the sample of cells exposed to the agent and in a sample of cells that is not exposed to the agent; and

c) determining that the cells will be responsive to the agent when genes in the sample of cells not exposed to the agent.

This embodiment of the methods of the present invention involves the step of exposing the cells to an agent. The method used for exposing the reproductive cells to the agent will be based primarily on the source and nature of the cells and the agent being tested. The contacting can be performed in vitro or in vivo, in a patient being treated/evaluated or in animal model of a reproductive disorder. For cells and cell lines and chemical compounds, exposing the cells involves contacting the cells with the compound, such as in tissue culture media. A skilled artisan can readily adapt an appropriate procedure for contacting cells with any particular agent or combination of agents.

As discussed above, the identified TNF & IFN stimulated genes of the invention can be used as markers to monitor an TNF & IFN influenced cellular process or response. For example, exposure to TNF & IFN can increase the resistance of cells to viral infection. Accordingly, by monitoring the expression of TNF & IFN stimulated genes one can assess whether a viral affected tissue has become reponsive or refractory to an ongoing treatment. When viral affected tissue is no longer responding to a treatment the expression profile of the viral affected tissue will change: the level of expression of one or more of the TNF & IFN stimulated genes will decrease.

In such a use, the invention provides methods for determining whether an treatment should be continued in a patient, comprising the steps of

a) obtaining a first sample of tissue from a patient undergoing therapy at a first time and obtaining a second sample of cells from the patient at a second later time;

b) determining the level of expression of one or more genes TNF & IFN stimulated genes (Table 1 and 2) in the first and second samples of cells; and

c) discontinuing treatment when the expression of one or more TNF & IFN stimulated genes is lower in the second sample of cells than in the first sample of cells.

As used here, a patient refers to any subject undergoing treatment for a disease or disorder involving viral infection or activity. The preferred subject will be a human patient undergoing TNF & IFN therapy.

This embodiment of the present invention relies on comparing two or more samples obtained from a patient undergoing TNF & IFN therapy. In general, it is preferable to obtain a first sample from the patient prior to beginning therapy and one or more samples during treatment. In such a use, a baseline of expression prior to therapy is determined and then changes in the baseline state of expression is monitored during the course of therapy. Alternatively, two or more successive sample obtained during treatment can be used without the need of a pre-treatment baseline sample. In such a use, the first sample obtained from the subject is used as a baseline for determining whether the expression of a particular gene is increasing or decreasing.

In general, when monitoring the effectiveness of a therapeutic treatment, two or more samples from the patient are examined. Preferably, three or more successively obtained samples are used, including at least one pretreatment sample.

### Kits Containing Reagents for Conducting the Methods of the Present Invention

The present invention further provides kits comprising compartmentalized containers comprising reagents for detecting one or more, preferably two or more, of the TNF & IFN stimulated genes of the present invention. As used herein a kit is defined as a prepackaged set of containers into which reagents are placed. The reagents included in the kit comprise probes/primers and/or antibodies for use in detecting TNF & IFN stimulated gene expression. In addition, the kits of the present invention may preferably contain instructions which describe the use of the kit. Such kits can be conveniently used, e.g., in clinical settings, to diagnose patients exhibiting, e.g., symptoms of a disorder associated with an TNF & IFN influenced cellular process response.

### Further Characterization of TNF & IFN Stimulated Genes

TNF & IFN stimulated genes of the present invention can be further characterized using techniques known to those skilled in the art to yield more information regarding potential targets for the therapeutic modulation of a TNF & IFN influenced cellular process or response, e.g., increased viral resistance. For example, characterization of the identified genes can yield information regarding the biological function of the identified genes.

Specifically, any of the TNF & IFN stimulated genes whose further characterization indicates that a modulation of the gene's expression or a modulation of the gene product's activity modulate an TNF & IFN influence cellular process or response are designated “target genes.” Target genes and target gene products can be used to identify therapeutics agents. An TNF & IFN stimulated gene whose further characterization indicates that it does not modulate an TNF & IFN influenced cellular process or response, but whose expression pattern contributes to a gene expression pattern correlative of, an TNF & IFN influenced cellular process or response cannot serve as a target gene. Such genes can be used as diagnostic markers and as markers for assessing or monitoring an TNF & IFN influenced cellular process or response.

A variety of techniques can be utilized to further characterize the genes herein identified. First, the nucleotide sequence of the identified genes, obtained by standard techniques well known to those of skill in the art, can be used to further characterize such genes. For example, the sequence of the identified genes can reveal homologies to one or more known sequence motifs that can yield information regarding the biological function of the identified gene product.

Second, an analysis of the tissue and/or cell type distribution of the mRNA produced by the identified genes can be conducted, utilizing standard techniques well known to those of skill in the art. Such techniques can include, for example, Northern analyses, RT-coupled PCR and RNase protection techniques. Such analyses can be used to determine whether cells within a given tissue express the identified gene. Such an analysis can also provide information regarding the biological function of an identified gene.

Third, the sequences of the identified genes can be used, utilizing standard techniques, to place the genes onto genetic maps, e.g., mouse (Copeland and Jenkins 1991, Trends in Genetics 7:113-118) and human genetic maps (Cohen et al., 1993, Nature 366:698-701). Such mapping information can yield information regarding the genes' importance to human disease by, for example, identifying genes that map within a genetic region to which predisposition to viral diseases, disorders, infection and/or resistance also maps.

Fourth, the biological function of the identified genes can be more directly assessed by utilizing relevant in vivo and in vitro systems. In vivo systems can include, but are not limited to, animal systems that naturally exhibit symptoms of a disorder of interest, e.g., an immune disorder or a proliferative disorder or ones that have been engineered to exhibit such symptoms. The role of identified gene products can be determined by transfecting cDNAs encoding these gene products into appropriate cell lines and analyzing the effect of the gene product on the cells.

In further characterizing the biological function of the identified genes, the expression of these genes can be modulated within the in vivo and/or in vitro systems, i.e., either over-expressed or under-expressed, and the subsequent effect on the system then assayed. Alternatively, the activity of the product of the identified gene can be modulated by either increasing or decreasing the level of activity in the in vivo and/or in vitro system of interest, and assessing the effect of such modulation. The information obtained through such characterizations can suggest relevant methods for the modulation of an TNF & IFN influenced cellular response or process, e.g., increased viral resistance. For example, treatment can include a modulation of gene expression and/or gene product activity. Characterization procedures such as those described herein can indicate where such modulation should involve an increase or a decrease in the expression or activity of the gene or gene product of interest.

### Identification of Compounds that Interact with a Target Gene Product

The following assays are designed to identify compounds that bind to target gene products, compounds that bind to other cellular proteins that interact with a target gene product, and compounds that interfere with the interaction of the target gene product with other cellular proteins. Such compounds can include, but are not limited to, other cellular proteins, natural products and small chemical molecules. Specifically, such compounds can include, but are not limited to, peptides, such as, for example, soluble peptides, including, but not limited to Ig-tailed fusion peptides, comprising extracellular portions of target gene product transmembrane receptors, and members of random peptide libraries (see, e.g., Lam et al., 1991, Nature 354:82-84; Houghton et al., 1991, Nature 354:84-86), made of D-and/or L-configuration amino acids, phosphopeptides (including, but not limited to, members of random or partially degenerate phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab)2and FAb expression library fragments, and epitope-binding fragments thereof), and small organic or inorganic molecules.

Compounds identified via assays such as those described herein can be useful, for example, in elaborating the biological function of the target gene product, and for modulating viral resistance or infection. For example, for TNF & IFN stimulated genes that are target genes, compounds that decrease the level of expression of the gene or the activity of the encoded protein, are potential therapeutic agents for viral vector delivery. Conversely, compounds that increase the level of expression of the gene or the activity of the encoded protein, are potential therapeutic agents for viral disorders requiring increased resistance.

### Screening Assays for Compounds and Cellular Proteins that Bind to a Target Gene Product

In vitro systems can be designed to identify compounds capable of binding the target gene products of the invention. Compounds thus identified can be used to modulate the activity of target gene products in a therapeutic protocol, to elaborate the biological function of the target gene product, or to identify compounds that disrupt normal target gene interactions. The preferred targets genes/products used in this embodiment are the TNF & IFN stimulated genes of the present invention.

The principle of the assays used to identify compounds that bind to the target gene product involves preparing a reaction mixture of the target gene protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring target gene product or the test substance onto a solid phase and detecting target gene product/test compound complexes anchored on the solid phase at the end of the reaction. In one embodiment of such a method, the target gene product can be anchored onto a solid surface, and the test compound, that is not anchored, can be labeled, either directly or indirectly.

In practice, microliter plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously immobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the immobilized component (the antibody, in turn, can be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for target gene or the test compound to anchor any complexes formed in solution, and-a labeled antibody specific for the other component of the possible complex to detect anchored complexes.

Any method suitable for detecting protein-protein interactions can be employed for identifying novel target product-cellular or extracellular protein interactions. In such a case, the target gene serves as the known “bait” gene.

### Assays for Compounds that Interfere with the Binding of a Target Gene Product to a Second Cellular Protein

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be usefull in regulating the activity of the target-gene product. Such compounds can include, but are not limited to molecules such as antibodies, peptides, and small molecules.

The basic principle of the assay systems used to identify compounds that interfere with the interaction between the target gene product and its cellular or extracellular binding partner or partners involves preparing a reaction mixture containing the target gene product, and the binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

The assay for compounds that interfere with the interaction of the target gene products and binding partners can be conducted in a heterogeneous or homogeneous format. Heterogeneous assays involve anchoring either the target gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance; i.e., by adding the test substance to the reaction mixture prior to or simultaneously with the target gene product and interactive cellular or extracellular binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the target gene product or the interactive cellular or extracellular binding partner, is anchored onto a solid surface, while the non-anchored species is labeled, either directly or indirectly. In practice, microtitre plates are conveniently utilized. The anchored species can be immobilized by noncovalent or covalent attachments. Non-covalent attachment can be accomplished simply by coating the solid surface with a solution of the target gene product or binding partner and drying. Alternatively, an immobilized antibody specific for the species to be anchored can be used to anchor the species to the solid surface. The surfaces can be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the nonimmobilized species is pre-labeled, the detection of label immobilized on the surface indicates that complexes were formed. Where the non-immobilized species is not prelabeled, an indirect label can be used to detect complexes anchored on the surface; e.g., using a labeled antibody specific for the initially non-immobilized species (the antibody, in turn, can be directly labeled or indirectly labeled with a labeled anti-Ig antibody). Depending upon the order of addition of reaction components, test compounds that inhibit complex formation or that disrupt preformed complexes can be detected. Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-cellular or extracellular binding partner interaction can be identified.

### Assays Based on Target Gene Product Activity

The present invention further provides methods for identifying new therapeutic agents, or combinations of therapeutic agents, that modulate the activity or expression of one or more the gene products encoded the TNF & IFN stimulated genes of the invention. Specifically, the activity of the proteins encoded by the genes of the present invention can be used as a basis for identifying agents which can be used to modulate an TNF & IFN influenced cellular process or response, e.g. increased viral resistance. For example, by blocking the activity of one or more of the proteins encoded by TNF & IFN stimulated genes of the invention, cells will become sensitive to treatment with an agent that the unmodified cells were resistant to.

The choice of assay format will be based primarily on the nature and type of protein being assayed. A skilled artisan can readily adapt protein activity assays for use in the present invention with the genes identified herein.

### Modulation of an TNF & IFN Influenced Cellular Process or Response by Modulation of TNF & IFN Stimulated Genes or Gene Products

TNF & IFN influenced cellular processes and responses, e.g., increased viral resistance, can be modulated by modulating the expression of a target gene or the activity of a target gene product. The modulation can be of a positive or negative nature, depending on the specific situation involved.

“Negative modulation,” refers to a reduction in the level and/or activity of target gene product relative to the level and/or activity of the target gene product in the absence of the modulatory treatment.

“Positive modulation,” refers to an increase in the level and/or activity of target gene product relative to the level and/or activity of target gene product in the absence of modulatory treatment.

It is possible that a disorder associated with an TNF & IFN influenced cellular process or response, can be caused, at least in part, by an abnormal level of a target gene product, or by the presence of a gene product exhibiting abnormal activity. As such, the reduction in the level and/or activity of such gene products would bring about the amelioration of the disorder. Alternatively, it is possible that such disorders can be brought about, at least in part, by the absence or reduction of the level of target gene expression, or a reduction in the level of a gene product's activity. As such, an increase in the level of gene expression and/or the activity of such gene products would bring about the amelioration of the disorder. As discussed, above, successful treatment of various disorders can be brought about by techniques that serve to increase the expression or activity of target gene products (i.e., the product of TNF & IFN stimulated genes that are target genes).

For example, compounds, e.g., an agent identified using an assays described above, that proves to exhibit positive modulatory activity, can be used in accordance with the invention to prevent and/or ameliorate symptoms of a disorder associated with an TNF & IFN influences cellular process or response, e.g., increased viral infection. Such molecules can include, but are not limited to peptides, phosphopeptides, small organic or inorganic molecules, or antibodies (including, for example, polyclonal, monoclonal, humanized, anti-idlotypic, chieric or single chain antibodies, and FAb, F(ab′)₂ and FAb expression library fragments, and epitope-binding fragments thereof.

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity.

Among the compounds that can be used to reduce or inhibit either wild type, or if appropriate, mutant target gene activity are antisense, ribozyme, and triple helix molecules. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred.

Ribozymes are enzymatic RNA molecules capable of catalyzing the specific cleavage of RNA. (For a review, see, for example, Rossi, 1994, Current Biology 4:469-471.) The mechanism of ribozyme action involves sequence specific hybridization of the ribozyme molecule to complementary target RNA, followed by a endonucleolytic cleavage. The composition of ribozyme molecules must include one or more sequences complementary to the target gene mRNA and must include the well-known catalytic sequence responsible for mRNA cleavage. For this sequence, see U.S. Pat. No. 5,093,246, that is incorporated by reference herein in its entirety. As such within the scope of the invention are engineered hammerhead motif ribozyme molecules that specifically and efficiently catalyze endonucleolytic cleavage of RNA sequences encoding target gene proteins.

Specific ribozyme cleavage sites within any potential RNA target are initially identified by scanning the molecule of interest for ribozyme cleavage sites that include the following sequences, GUA, GUU, and GUC. Once identified, short RNA sequences of between 15 and 20 ribonucleotides corresponding to the region of the target gene containing the cleavage site can be evaluated for predicted structural features, such as secondary structure, that can render the oligonucleotide sequence unsuitable. The suitability of candidate sequences can also be evaluated by testing their accessibility to hybridization with complementary oligonucleotides, using ribonuclease protection assays.

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, that generally require sizeable stretches of either purines or pyrimidines to be present on one strand of a duplex. Nucleotide sequences can be pyrimidine-based, that will result in TAT and CGC′ triplets across the three associated strands of the resulting triple helix. The pyrinidine-rich molecules provide base complementarily to a purine-rich region of a single strand of the duplex in a parallel orientation to that strand. In addition, nucleic acid molecules can be chosen that are purine-rich, for example, contain a stretch of G residues. These molecules will form a triple helix with a DNA duplex that is rich in GC pairs, in that the majority of the purine residues are located on a single strand of the targeted duplex, resulting in GGC triplets across the three strands in the triplex. Alternatively, the potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to reduce or inhibit mutant gene expression, it is possible that the technique utilized can also efficiently reduce or inhibit the transcription (triple helix) and/or translation (antisense, ribozyme) of mRNA produced by normal target gene alleles such that the possibility can arise wherein the concentration of normal target gene product present can be lower than is necessary for a normal phenotype. In such cases, to ensure that substantially normal levels of target gene activity are maintained, nucleic acid molecules that encode and express target gene polypeptides exhibiting normal target gene activity can be introduced into cells via gene therapy method. Alternatively, in instances in that the target gene encodes an extracellular protein, it can be preferable to co-administer normal target gene protein into the cell or tissue in order to maintain the requisite level of cellular or tissue target gene activity.

Anti-sense RNA and DNA, ribozyme and triple helix molecules of the invention can be prepared by any method known in the art for the synthesis of DNA and RNA molecules. These include techniques for chemically synthesizing oligodeoxyribonucleotides and oligoribonucleotides well known in the art such as, for example, solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules can be introduced as a means of increasing intracellular stability and half-life. Possible modifications include but are not limited to the addition of flanking sequences of ribo- or deoxynucleotides to the 5′ and/or 3′ ends of the molecule or the use of phosphorothioate or 2′ O-methyl rather than phosphodiesterase linkages within the oligodeoxyribonucleotide backbone.

Antibodies can be generated that are both specific for target gene product and that reduce target gene product activity. Such antibodies may, therefore, by administered in instances whereby negative modulatory techniques are appropriate. Antibodies can be generated using standard techniques against the proteins themselves or against peptides corresponding to portions of the proteins. The antibodies include but are not limited to polyclonal, monoclonal, Fab fragments, single chain antibodies, chimeric antibodies, and the like.

In instances where the target gene protein to that the antibody is directed is intracellular and whole antibodies are used, internalizing antibodies can be preferred. However, lipofectin or liposomes can be used to deliver the antibody or a fragment of the Fab region that binds to the target gene epitope into cells. Where fragments of the antibody are used, the smallest inhibitory fragment that binds to the target protein's antibinding domain is preferred. For example, peptides having an amino acid sequence corresponding to the domain of the variable region of the antibody that binds to the target gene protein can be used. Such peptides can be synthesized chemically or produced via recombinant DNA technology using methods well known in the art (e.g., see Creighton, 1983, supra; and Sambrook et al., 1989, supra). Alternatively, single chain neutralizing antibodies that bind to intracellular target gene product epitopes can also be administered. Such single chain antibodies can be administered, for example, by expressing nucleotide sequences encoding single-chain antibodies within the target cell population by utilizing, for example, techniques such as those described in Marasco et al. (1993, Proc. Natl. Acad. Sci. USA 90:7889-7893).

### Therapeutic Treatment

The identified compounds that modulate target gene expression, synthesis and/or activity can be administered to a patient at therapeutically effective doses to prevent, treat or ameliorate a disorder associated with an TNF & IFN influenced cellular process or response. A therapeutically effective dose refers to that amount of the compound sufficient to result in amelioration of symptoms of the disorder.

### Effective Dose

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50 Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans: Levels in plasma can be measured, for example, by high performance liquid chromatography.

### Formulations and Use

Pharmaceutical compositions for use in accordance with the present invention can be formulated in conventional manner using one or more physiologically acceptable carriers or excipients.

Thus, the compounds and their physiologically acceptable salts and solvates can be formulated for administration by inhalation or insufflation (either through the mouth or the nose) or oral, buccal, parenteral or rectal administration.

For oral administration, the pharmaceutical compositions can take the form of, for example, Tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The Tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of Tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt. The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

### EXAMPLE

### Identification of TNF & IFN Stimulated Genes

A mouse CDNA array from Incyte, Inc. was used to analyze the expression profile of Y-1 (ATCC Accession No. CCL 79) cells. This transcriptional profile analysis led to the identification of the TNF and IFN stimulated genes of the invention.

Y1 cells were incubated with Ham's F-10 Nutrient Mixture Media supplemented with glutamine (1%), G418 geneyticin (80 ug/ml), penicillin-streptomycin (1%), fetal horse serum (15%), and fetal bovine serum (2.5%) and grown to confluency. Confluent cells were then exposed to TNF-α (R&D Systems) at 1 ug/ml and IFN-γ (R&D Systems) at 1 ug/ml for 1 hour (see Table 1) and 16 hours (see Table 2). Total RNA was isolated from control or treated cells with ULTRASPEC™ RNA from Biotech Laboratories, Inc. All media and supplements were obtained from Gibco BRL Life Technologies.

The GEM (Gene Expression Microarray) technology uses the following steps to discover differences in gene expression between two messenger RNA (mRNA) samples. Small samples of cDNA were deposited on a glass surface and bonded to the glass. Subsequently, large portions from one half of the DNA's double strands are removed in order to activate the individual elements of the array, preparing them to react and bind to their uniquely matched DNA counterparts in the cells being tested.

Two mRNA samples were prepared and color labeled, since the GEM technology uses a color coding technique to discover the differences in gene expression between two mRNA samples. Messenger RNA was extracted from the normal or unaffected sample, and a fluorescent labeled cDNA probe is generated. The probe represents all of the genes expressed in the reference sample. Next, the mRNA was extracted from another sample. Typically, these are the affected cells (e.g., Y-1 cells): exposed to a drug or toxic substance or removed at a different time. The fluorescent labeling step was repeated to generate a second cDNA probe using a different color fluorescent molecule.

The two fluorescent probe samples were simultaneously applied to a single microarray, where they competitively react with the arrayed cDNA molecules. Following incubation, the microarray was rinsed, washing off those probe molecules that did not find their cDNA counterpart. Each element of the GEM microarray was scanned for the first fluorescent color. The intensity of the fluorescence at each array element is proportional to the expression level of that gene in the sample. The scanning operation was repeated for the second fluorescent label. The ratio of the two fluorescent intensities provides a quantitative measurement of the relative gene expression level in the two cell samples. For example, if a microarray element shows no color, it indicates that the gene in that element was not expressed in either cell sample. If an element shows a single color, it indicates that a labeled gene was expressed only in that cell sample. The appearance of both colors indicates that the gene was expressed in both cell samples.

The genes identified on the GEM array that are more highly expressed in Y-1 treated cells than control cells are listed in Tables 1 and 2 in order of decreasing differential expression. Each entry includes the I.M.A.G.E. Database Accession Number for the sequence. I.M.A.G.E. clones can be obtained from, e.g., the American Type Culture Collection (Manassas, Va.).

### TABLE 1

TNF & IFN-g 223 genes up-regulated in Y1 cells 16 hr

| Fold | Cont | TNF + IFNg | Gene name | Acc# |
|---|---|---|---|---|
| 2.2 | 50 | 109 | Acid beta glucosidase {IMAGE:331646} | W08086.1 |
| 2.1 | 76 | 157 | AMP deaminase 3 {IMAGE:750480} | AA389897.1 |
| 2.1 | 53 | 109 | Arachidonate 12-lipoxygenase, pseudogene 2 {IMAGE:335369} | W36511.1 |
| 2.4 | 115 | 281 | B-cell leukemia/lymphoma 3 {IMAGE:716976} | AA266002.1 |
| 2.1 | 83 | 172 | B-cell translocation gene 2, anti-proliferative {IMAGE:583186} | AA154848.1 |
| 2 | 53 | 105 | Cathelin-like protein {IMAGE:334755} | W17771.1 |
| 2 | 40 | 79 | CCAAT/enhancer binding protein alpha (C/EBP), related sequence 1 {IM | AA473938.1 |
| 2 | 54 | 106 | Coagulation factor II {IMAGE:350344} | W40725.1 |
| 2.2 | 40 | 88 | Crystallin, gamma S {IMAGE:483498} | AI893668.1 |
| 2.2 | 43 | 94 | Cytochrome P450, 2b13, phenobarbitol inducible, type c {IMAGE:31628 | AI325330.1 |
| 2.1 | 68 | 141 | EST {IMAGE:474319} | AA038807.1 |
| 2.6 | 409 | 1083 | ESTs {IMAGE:315676} | W09957.1 |
| 2.4 | 434 | 1047 | ESTs {IMAGE:316914} | W11926.1 |
| 2.1 | 51 | 106 | ESTs {IMAGE:317515} | W34080.1 |
| 2.1 | 151 | 316 | ESTs {IMAGE:318969} | W34482.1 |
| 2.2 | 37 | 82 | ESTs {IMAGE:330233} | W14737.1 |
| 2.3 | 66 | 154 | ESTs {IMAGE:334410} | W18278.1 |
| 2.7 | 245 | 663 | ESTs {IMAGE:350182} | W34722.1 |
| 2.1 | 53 | 111 | ESTs {IMAGE:372368} | W53544.1 |
| 2 | 258 | 517 | ESTs {IMAGE:373112} | W62546.1 |
| 2 | 65 | 133 | ESTs {IMAGE:385744} | W64241.1 |
| 2.1 | 65 | 136 | ESTs {IMAGE:403413} | W82905.1 |
| 2.9 | 59 | 170 | ESTs {IMAGE:404589} | W82894.1 |
| 2 | 544 | 1090 | ESTs {IMAGE:419146} | W88005.1 |
| 2.1 | 101 | 217 | ESTs {IMAGE:427480} | AA002452.1 |
| 2 | 324 | 663 | ESTs {IMAGE:436999} | AA002783.1 |
| 2.3 | 69 | 159 | ESTs {IMAGE:459562} | AA027381.1 |
| 2.1 | 61 | 131 | ESTs {IMAGE:466371} | AA033504.1 |
| 2.4 | 121 | 285 | ESTs {IMAGE:467873} | AA036517.1 |
| 2 | 54 | 109 | ESTs {IMAGE:478040} | AI322694.1 |
| 2.1 | 60 | 124 | ESTs {IMAGE:478157} | AA049610.1 |
| 2.7 | 291 | 772 | ESTs {IMAGE:482641} | AA061982.1 |
| 2.1 | 77 | 158 | ESTs {IMAGE:483381} | AI552474.1 |
| 2 | 69 | 135 | ESTs {IMAGE:493212} | AI595201.1 |
| 3.2 | 64 | 204 | ESTs {IMAGE:571219} | AA107718.1 |
| 2.2 | 95 | 208 | ESTs {IMAGE:577555} | AA138025.1 |
| 6.2 | 38 | 234 | ESTs {IMAGE:583566} | AA143986.1 |
| 4.6 | 194 | 896 | ESTs {IMAGE:595951} | AA140542.1 |
| 9.7 | 50 | 484 | ESTs {IMAGE:596863} | AA145136.1 |
| 2.5 | 41 | 102 | ESTs {IMAGE:597493} | AA154419.1 |
| 4.9 | 26 | 128 | ESTs {IMAGE:598824} | AA168416.1 |
| 2.1 | 46 | 95 | ESTs {IMAGE:617402} | AA174434.1 |
| 2.1 | 161 | 341 | ESTs {IMAGE:618611} | AA174941.1 |
| 17.1 | 22 | 376 | ESTs {IMAGE:619723} | AA172624.1 |
| 3.6 | 36 | 129 | ESTs {IMAGE:637888} | AA189578.1 |
| 2 | 50 | 99 | ESTs {IMAGE:640013} | AA198524.1 |
| 2.1 | 82 | 171 | ESTs {IMAGE:669765} | AA230444.1 |
| 3.7 | 266 | 995 | ESTs {IMAGE:678854} | AA250462.1 |
| 3.4 | 209 | 713 | ESTs {IMAGE:751826} | AA396152.1 |
| 3.4 | 41 | 138 | ESTs {IMAGE:761264} | AA388221.1 |
| 2.2 | 53 | 118 | ESTs {IMAGE:775033} | AA387356.1 |
| 2.3 | 68 | 156 | ESTs {IMAGE:775092} | AA386832.1 |
| 2 | 47 | 96 | ESTs {IMAGE:775188} | AA387164.1 |
| 2.1 | 49 | 102 | ESTs {IMAGE:775208} | AA387199.1 |
| 2 | 45 | 88 | ESTs {IMAGE:792921} | AA415579.1 |
| 2 | 61 | 122 | ESTs {IMAGE:793692} | AA415691.1 |
| 2 | 53 | 107 | ESTs {IMAGE:804793} | AA472833.1 |
| 2.3 | 104 | 238 | ESTs {IMAGE:805129} | AA473234.1 |
| 3.4 | 45 | 153 | ESTs {IMAGE:805386} | AA473205.1 |
| 4 | 393 | 1563 | ESTs {IMAGE:817924} | AA466508.1 |
| 2.4 | 61 | 146 | ESTs {IMAGE:818790} | AA467382.1 |
| 2 | 38 | 75 | ESTs {IMAGE:831741} | AA444941.1 |
| 2.3 | 38 | 87 | ESTs {IMAGE:833477} | AA465972.1 |
| 2.6 | 62 | 159 | ESTs {IMAGE:834617} | AA467546.1 |
| 2.1 | 48 | 100 | ESTs {IMAGE:835536} | AA422515.1 |
| 2.1 | 43 | 91 | ESTs {IMAGE:836133} | AA426897.1 |
| 3.5 | 59 | 209 | ESTs {IMAGE:837565} | AA432818.1 |
| 2 | 47 | 95 | ESTs {IMAGE:845839} | AA416091.1 |
| 2 | 80 | 158 | ESTs {IMAGE:872986} | AA466235.1 |
| 2.1 | 99 | 207 | ESTs {IMAGE:876106} | AA475528.1 |
| 2 | 50 | 98 | ESTs {IMAGE:904836} | AA522117.1 |
| 3.6 | 118 | 426 | ESTs {IMAGE:945014} | AA537234.1 |
| 2.8 | 48 | 132 | ESTs {IMAGE:961094} | AA547202.1 |
| 2.1 | 147 | 310 | ESTs, Highly similar to CYSTATHIONINE GAMMA-LYASE [Homo sapier | AI892342.1 |
| 2.5 | 59 | 148 | ESTs, Highly similar to CYSTATHIONINE GAMMA-LYASE [Homo sapier | AA245993.1 |
| 5.7 | 55 | 313 | ESTs, Highly similar to HYPOTHETICAL 103.5 KD PROTEIN IN RAD26-( | AI639807.1 |
| 4 | 580 | 2318 | ESTs, Highly similar to INTERFERON-INDUCIBLE PROTEIN [Rattus no | AA048837.1 |
| 6.3 | 40 | 253 | ESTs, Highly similar to LYSYL-TRNA SYNTHETASE [Cricetulus longica | AA175815.1 |
| 4.1 | 165 | 683 | ESTs, Highly similar to NUCLEAR FACTOR NF-KAPPA-B P100 SUBUNI | AI385509.1 |
| 3 | 149 | 449 | ESTs, Highly similar to SERINE HYDROXYMETHYLTRANSFERASE, MI | AA208877.1 |
| 2.2 | 152 | 336 | ESTs, Highly similar to UTP-GLUCOSE-1-PHOSPHATE URIDYLYLTRAI | AA473123.1 |
| 2.3 | 389 | 888 | ESTs, Highly similar to 54 kDa oligoadenylate synthetase-like protein p | AA123007.1 |
| 2.3 | 49 | 111 | ESTs, Highly similar to FLASH [M. musculus] {IMAGE:833829} | AA412925.1 |
| 2.8 | 218 | 603 | ESTs, Highly similar to KIAA0650 protein [H. sapiens] {IMAGE:621629} | AA183687.1 |
| 3.2 | 62 | 197 | ESTs, Highly similar to major vault protein [R. norvegicus] {IMAGE:6395 | AA200827.1 |
| 2 | 67 | 131 | ESTs, Highly similar to molybdopterin-synthase large subunit [M. musc | AI450527.1 |
| 2.2 | 45 | 100 | ESTs, Highly similar to myomegalin [R. norvegicus] {IMAGE:315787} | W12392.1 |
| 2.2 | 50 | 110 | ESTs, Highly similar to NY-REN-2 antigen [H. sapiens] {IMAGE:945689} | AA546195.1 |
| 2 | 47 | 92 | ESTs, Highly similar to pirin [H. sapiens] {IMAGE:332396} | W08720.1 |
| 2.6 | 142 | 373 | ESTs, Highly similar to probable nocturnin protein [M. musculus] {IMAG | AA051133.1 |
| 2.6 | 71 | 185 | ESTs, Highly similar to putative transcription factor [H. sapiens] {IMAGE | AA467436.1 |
| 2 | 208 | 410 | ESTs, Highly similar to unknown [H. sapiens] {IMAGE:336564} | AI390954.1 |
| 2.3 | 132 | 298 | ESTs, Highly similar to unknown [H. sapiens] {IMAGE:492447} | AA097194.1 |
| 3.2 | 87 | 277 | ESTs, Moderately similar to ACTIN-LIKE PROTEIN 14D [Drosophila mel | AA451385.1 |
| 2 | 46 | 92 | ESTs, Moderately similar to GEPHYRIN [Rattus norvegicus] {IMAGE:61 | AI893727.1 |
| 2 | 49 | 100 | ESTs, Moderately similar to C11D2.4 [C. elegans] {IMAGE:441807} | AA014053.1 |
| 2 | 50 | 101 | ESTs, Moderately similar to homocysteine respondent protein HCYP2 [I | W20810.1 |
| 2 | 337 | 665 | ESTs, Moderately similar to KIAA0651 protein [H. sapiens] {IMAGE:6952 | AA220487.1 |
| 2.1 | 68 | 140 | ESTs, Moderately similar to probable ligand-binding protein RYD5 [R. n | AA412821.1 |
| 2.1 | 61 | 130 | ESTs, Moderately similar to SARCOLIPIN [H. sapiens] {IMAGE:318692} | W11587.1 |
| 2.9 | 61 | 174 | ESTs, Moderately similar to unknown [H. sapiens] {IMAGE:761038} | AA387579.1 |
| 2.1 | 59 | 124 | ESTs, Weakly similar to CYP2C39 [M. musculus] {IMAGE:367514} | AI427394.1 |
| 2.3 | 39 | 90 | ESTs, Weakly similar to cytohesin 2 [M. musculus] {IMAGE:779020} | AA413956.1 |
| 2.1 | 85 | 177 | ESTs, Weakly similar to CYTOSKELETAL P17 PROTEIN [Dictyostelium | AA017875.1 |
| 2.2 | 59 | 131 | ESTs, Weakly similar to DUAL SPECIFICITY PROTEIN PHOSPHATASE 1 | AA119029.1 |
| 2.2 | 623 | 1381 | ESTs, Weakly similar to GARP PROTEIN PRECURSOR [H. sapiens] {IMA | AA048874.1 |
| 2.3 | 43 | 97 | ESTs, Weakly similar to gob-5 [M. musculus] {IMAGE:351090} | W41083.1 |
| 2.1 | 71 | 150 | ESTs, Weakly similar to HYPOTHETICAL 11.4 KD PROTEIN C13G6.04 I | W11535.1 |
| 2.2 | 45 | 98 | ESTs, Weakly similar to keratin 10, type I, cytoskeletal [H. sapiens] {IMA | W18457.1 |
| 3.1 | 310 | 950 | ESTs, Weakly similar to LYMPHOCYTE ANTIGEN LY-6A.2/LY-6E.1 PRE | AA472994.1 |
| 3.5 | 81 | 283 | ESTs, Weakly similar to lysophospholipase I [M. musculus] {IMAGE:596 | AA138854.1 |
| 2.5 | 705 | 1750 | ESTs, Weakly similar to mannose-binding lectin associated serine prot | AA209006.1 |
| 2.3 | 107 | 249 | ESTs, Weakly similar to mCAC [M. musculus] {IMAGE:350881} | W40994.1 |
| 2.6 | 81 | 212 | ESTs, Weakly similar to ORF YJL004c [S. cerevisiae] {IMAGE:933229} | AA541946.1 |
| 2 | 57 | 113 | ESTs, Weakly similar to Pro-Pol-dUTPase polyprotein [M. musculus] {IM | AA518000.1 |
| 2.1 | 180 | 371 | ESTs, Weakly similar to SDP8 [M. musculus] {IMAGE:747180} | AA260397.1 |
| 2.2 | 66 | 143 | ESTs, Weakly similar to similar to alpha/beta hydrolase fold [C. elegans] | W09936.1 |
| 2.1 | 76 | 161 | ESTs, Weakly similar to similar to leucyl-tRNA synthetase [C. elegans] {I | AA397172.1 |
| 2.3 | 566 | 1300 | ESTs, Weakly similar to tazarotene-induced gene 2 [H. sapiens] {IMAGE | AA220505.1 |
| 2.3 | 315 | 711 | ESTs, Weakly similar to Trif [M. musculus] {IMAGE:523380} | AA080169.1 |
| 2.4 | 298 | 724 | ESTs, Weakly similar to UBIQUITIN-CONJUGATING ENZYME E2-16 KD | AA050167.1 |
| 2.5 | 591 | 1467 | Extracellular matrix protein 1 {IMAGE:874833} | AA474897.1 |
| 2.2 | 428 | 926 | Glutamate oxaloacetate transaminase 1, soluble {IMAGE:481381} | AA060494.1 |
| 2.2 | 45 | 97 | Hemochromatosis {IMAGE:313224} | W11889.1 |
| 12.5 | 37 | 464 | Histocompatibility 2, class II antigen A, alpha {IMAGE:747378} | AA272807.1 |
| 3.1 | 62 | 190 | Histocompatibility 2, class II antigen A, beta 1 {IMAGE:618271} | AA175329.1 |
| 3.9 | 59 | 231 | Histocompatibility 2, class II, locus Ma {IMAGE:669933} | AA230649.1 |
| 2 | 329 | 660 | Histocompatibility 2, K region {IMAGE:330336} | W14540.1 |
| 2.2 | 143 | 314 | Histocompatibility 2, Q region locus 7 {IMAGE:596447} | AA122791.1 |
| 2.2 | 311 | 688 | Histocompatibility 2, Q region locus 7 {IMAGE:637849} | AA186012.1 |
| 2 | 560 | 1133 | Histocompatibility 2, T region locus 22 {IMAGE:920425} | AA538511.1 |
| 2.1 | 107 | 225 | Homolog of human MLLT2 unidentified gene {IMAGE:750930} | AA289139.1 |
| 6.7 | 36 | 240 | Interferon activated gene 203 {IMAGE:620541} | AA174447.1 |
| 4.1 | 273 | 1123 | Interferon regulatory factor 1 {IMAGE:777393} | AA276697.1 |
| 3.4 | 801 | 2763 | Lymphocyte antigen 6 complex {IMAGE:580715} | AA145865.1 |
| 2.7 | 539 | 1462 | Lymphocyte antigen 6 complex, locus C {IMAGE:425855} | AA000712.1 |
| 5.8 | 37 | 216 | M. musculus mRNA for caspase-11 {IMAGE:618272} | AA175651.1 |
| 2.5 | 534 | 1329 | M. musculus mRNA for mTGIF protein {IMAGE:474339) | AA038657.1 |
| 3.3 | 322 | 1066 | M .musculus mRNA for mTGIF protein {IMAGE:722623) | AA260654.1 |
| 2.3 | 61 | 140 | M. musculus spasmolytic polypeptide (SP) mRNA {IMAGE:314078) | W09829.1 |
| 2.1 | 47 | 99 | Metallothionein 2 {IMAGE:427339} | AA003303.1 |
| 49.5 | 62 | 3070 | Monokine induced by gamma interferon {IMAGE:874758} | AA472492.1 |
| 3 | 557 | 1655 | Mouse chromatin nonhistone high mobility group protein (HGM-I(Y), co | AA538243.1 |
| 2.3 | 94 | 216 | Mus musculus A10 mRNA, partial cds {IMAGE:333376} | W15888.1 |
| 2.2 | 401 | 902 | Mus musculus A10 mRNA, partial cds {IMAGE:385441} | W61383.1 |
| 2 | 72 | 145 | Mus musculus Ankhzn mRNA, complete cds {IMAGE:894596} | AA517717.1 |
| 2 | 349 | 710 | Mus musculus asparagine synthetase mRNA, complete cds {IMAGE:33 | W29492.1 |
| 2 | 147 | 287 | Mus musculus BS4 peptide mRNA, complete cds {IMAGE:459091} | AA024028.1 |
| 2 | 162 | 317 | Mus musculus CIPER protein (Ciper) mRNA, complete cds {IMAGE:355 | W47752.1 |
| 2.1 | 64 | 137 | Mus musculus classII MHC transactivator CIITA mRNA, complete cds {I | AA178010.1 |
| 2.1 | 400 | 836 | Mus musculus G-protein-like LRG-47 mRNA, complete cds {IMAGE:651 | AA214784.1 |
| 2.1 | 36 | 74 | Mus musculus IER5 (ler5) mRNA, complete cds {IMAGE:643745} | AA203798.1 |
| 2.4 | 57 | 136 | Mus musculus interferon regulatory factor 5 (mirf5) mRNA, complete cd | AA185025.1 |
| 53.7 | 29 | 1558 | Mus musculus interferon-induced guanylate binding protein GBP-2 (Gb | AA153021.1 |
| 2.4 | 44 | 107 | Mus musculus intersectin-EH binding protein lbp2 mRNA, partial cds {II | AA413761.1 |
| 2.1 | 141 | 291 | Mus musculus ion channel homolog RIC mRNA, complete cds {IMAGE: | W98807.1 |
| 2.2 | 59 | 127 | Mus musculus major histocompatibility complex region NG27, NG28, R | W13905.1 |
| 2.5 | 36 | 90 | Mus musculus mRNA for DNA helicase Q1, complete cds {IMAGE:33049 | AI390823.1 |
| 6.4 | 39 | 249 | Mus musculus mRNA for GTPI protein {IMAGE:621346} | AA178613.1 |
| 20.2 | 35 | 708 | Mus musculus mRNA for IIGP protein {IMAGE:747208} | AA260490.1 |
| 20.9 | 50 | 1044 | Mus musculus mRNA for IIGP protein {IMAGE:764385} | AA277451.1 |
| 2 | 42 | 82 | Mus musculus mRNA for MD-1, complete cds {IMAGE:402964} | W80147.1 |
| 2 | 49 | 99 | Mus musculus peroxisomal biogenesis factor (Pex11a) mRNA, complet | AA237383.1 |
| 10 | 81 | 809 | Mus musculus putative purine nucleotide binding protein mRNA, compl | AA240404.1 |
| 2.3 | 128 | 288 | Mus musculus secreted carbonic anhydrase isozyme VI precursor, mRI | AI327498.1 |
| 2.5 | 535 | 1362 | Myelocytomatosis oncogene {IMAGE:441346} | AA009268.1 |
| 2.7 | 234 | 625 | Myeloid differentiation primary response gene 118 {IMAGE:386412} | W64388.1 |
| 3.2 | 520 | 1650 | MYELOID DIFFERENTIATION PRIMARY RESPONSE PROTEIN MYD116 | AA050417.1 |
| 2 | 352 | 719 | NAD-DEPENDENT METHYLENETETRAHYDROFOLATE DEHYDROGENA | W84014.1 |
| 3.2 | 507 | 1629 | Protease (prosome, macropain) 28 subunit, alpha {IMAGE:678768} | AA239485.1 |
| 2 | 83 | 162 | Protein tyrosine phosphatase, non-receptor type 2 {IMAGE:437517} | AA003599.1 |
| 2.2 | 48 | 105 | Public domain EST {IMAGE:314509} | W10776.1 |
| 2.2 | 49 | 110 | Public domain EST {IMAGE:314524} | W10780.1 |
| 2.3 | 53 | 122 | Public domain EST {IMAGE:330298} | W13940.1 |
| 2 | 53 | 106 | Public domain EST {IMAGE:350327} | W40724.1 |
| 2.4 | 82 | 194 | Public domain EST {IMAGE:353202} | W41810.1 |
| 2.1 | 53 | 109 | Public domain EST {IMAGE:367869} | W54520.1 |
| 2.1 | 42 | 88 | Public domain EST {IMAGE:372366} | W53543.1 |
| 2 | 63 | 128 | Public domain EST {IMAGE:374745} | W59461.1 |
| 2.1 | 39 | 83 | Public domain EST {IMAGE:390150} | W70677.1 |
| 2.2 | 41 | 89 | Public domain EST {IMAGE:403961} | W82923.1 |
| 2 | 131 | 256 | Public domain EST {IMAGE:439362} | AI466638. 1 |
| 2.1 | 47 | 101 | Public domain EST {IMAGE:442201} | AA013653.1 |
| 2.2 | 58 | 129 | Public domain EST {IMAGE:456831} | AA023545.1 |
| 2 | 57 | 114 | Public domain EST {IMAGE:459599} | AA027528.1 |
| 2 | 96 | 196 | Public domain EST {IMAGE:463183} | AA030360.1 |
| 2 | 50 | 101 | Public domain EST {IMAGE:465170} | AA030746.1 |
| 2.3 | 48 | 109 | Public domain EST {IMAGE:466376} | AA032359.1 |
| 2 | 44 | 90 | Public domain EST {IMAGE:478309} | AA049436.1 |
| 2 | 51 | 101 | Public domain EST {IMAGE:479480} | AA049260.1 |
| 4.3 | 58 | 252 | Public domain EST {IMAGE:482196} | AA059908.1 |
| 2.3 | 78 | 176 | Public domain EST {IMAGE:482817} | AA062120.1 |
| 3.3 | 93 | 303 | Public domain EST {IMAGE:574439} | AA118291.1 |
| 4.7 | 108 | 507 | Public domain EST {IMAGE:596231} | AA122443.1 |
| 13.8 | 27 | 372 | Public domain EST {IMAGE:597020} | AA138757.1 |
| 4.4 | 121 | 536 | Public domain EST {IMAGE:617529} | AA174721.1 |
| 4.6 | 77 | 355 | Public domain EST {IMAGE:618199} | AA175243.1 |
| 2.1 | 66 | 138 | Public domain EST {IMAGE:619596} | AA175803.1 |
| 2 | 82 | 162 | Public domain EST {IMAGE:620030} | AA178100.1 |
| 3.5 | 93 | 324 | Public domain EST {IMAGE:621742} | AA177584.1 |
| 6.3 | 27 | 169 | Public domain EST {IMAGE:636640} | AA204588.1 |
| 2.2. | 41 | 89 | Public domain EST {IMAGE:652811} | AA217625.1 |
| 4 | 35 | 141 | Public domain EST {IMAGE:653161} | AI390695.1 |
| 3.1 | 169 | 521 | Public domain EST {IMAGE:733734} | AA272876.1 |
| 2.1 | 93 | 193 | Public domain EST {IMAGE:760824} | AA386627.1 |
| 2.8 | 47 | 130 | Public domain EST {IMAGE:760917} | AA386815.1 |
| 2.8 | 57 | 159 | Public domain EST {IMAGE:761009} | AA387552.1 |
| 2.4 | 62 | 147 | Public domain EST {IMAGE:774996} | AA387314.1 |
| 4.6 | 50 | 231 | Public domain EST {IMAGE:775154} | AI604749.1 |
| 2.2 | 44 | 97 | Public domain EST {IMAGE:778923} | AA414282.1 |
| 2 | 44 | 86 | Public domain EST {IMAGE:779453} | AA414212.1 |
| 2.2 | 67 | 146 | Public domain EST {IMAGE:805306} | AA473329.1 |
| 2.5 | 71 | 180 | Public domain EST {IMAGE:948845} | AA544859.1 |
| 2.7 | 86 | 229 | Recombining binding protein suppressor of hairless (Drosophila) {IMA( | W97158.1 |
| 2.3 | 39 | 88 | SAPK/Erk/kinase 1 {IMAGE:778459} | AA413508.1 |
| 7.4 | 116 | 859 | Signal transducer and activator of transcription 1 {IMAGE:749544} | AA388678.1 |
| 2.2 | 52 | 113 | Thymus cell antigen 1, theta {IMAGE:316409} | W13151.1 |
| 15.7 | 40 | 629 | Transporter 1, ABC (ATP binding cassette) {IMAGE:576775} | AA123837.1 |
| 2.3 | 45 | 104 | Transthyretin {IMAGE:335319} | W17647.1 |
| 7.7 | 198 | 1527 | TRYPTOPHANYL-TRNA SYNTHETASE {IMAGE:367765} | W53959.1 |
| 2.6 | 401 | 1028 | Tumor necrosis factor induced protein 2 {IMAGE:949472} | AA537672.1 |
| 2.2 | 80 | 175 | Zinc finger protein 35 {IMAGE:422121} | AI893996.1 |
| 2.3 | 82 | 190 | Zinc transporter 4 {IMAGE:643619} | AA203866.1 |

### TABLE 2

| Fold | Cont | Tg | Gene name | Acc# |
|---|---|---|---|---|
| 2.7 | 527 | 1434 | apoptosis Inhibitor 1 {IMAGE:640046} | AA197349.1 |
| 2.1 | 279 | 581 | B-cell leukemia/lymphoma 10 {IMAGE:355209} | W47752.1 |
| 2.5 | 2462 | 6113 | beta-2 microglobulin {IMAGE:572542} | AA109951.1 |
| 2.1 | 609 | 1263 | capping protein alpha 1 {IMAGE:779754} | AA414612.1 |
| 3.1 | 125 | 388 | caspase 11 {IMAGE:618272} | AA175651.1 |
| 2.1 | 1054 | 2215 | cell division cycle 42 homolog (S. cerevislae) {IMAGE:721518} | AA266975.1 |
| 3.4 | 321 | 1087 | ESTs {IMAGE:523814} | AA067146.1 |
| 3.4 | 190 | 639 | ESTs {IMAGE:583566} | AA143986.1 |
| 9.6 | 369 | 3541 | ESTs {IMAGE:595951} | AA140542.1 |
| 2 | 150 | 298 | ESTs {IMAGE:596556} | AA146363.1 |
| 3.9 | 165 | 647 | ESTs {IMAGE:596863} | AA145136.1 |
| 2.5 | 108 | 265 | ESTs {IMAGE:597031} | AA145466.1 |
| 5.6 | 148 | 827 | ESTs {IMAGE:619723} | AA172624.1 |
| 3.5 | 125 | 441 | ESTs {IMAGE:636640} | AA204588.1 |
| 2 | 556 | 1133 | ESTs {IMAGE:636827} | AA183302.1 |
| 5.7 | 366 | 2072 | ESTs {IMAGE:678854} | AA250462.1 |
| 3.1 | 306 | 954 | ESTs {IMAGE:751826} | AA396152.1 |
| 3.3 | 162 | 542 | ESTs {IMAGE:777379} | AA277040.1 |
| 2.5 | 368 | 928 | ESTs {IMAGE:876257} | AA476183.1 |
| 2 | 237 | 484 | ESTs, Highly similar to HYPOTHETICAL 103.5 KD PROTEIN IN RAD26-GI | AI639807.1 |
| 2.2 | 224 | 502 | ESTs, Highiy similar to INTERFERON-ACTIVATABLE PROTEIN 204 [Mus | AI591873.1 |
| 2.8 | 866 | 2458 | ESTs, Highly similar to INTERFERON-INDUCIBLE PROTEIN [Rattus norv | AA048837.1 |
| 2.1 | 243 | 511 | ESTs, Highly sImilar to AE-1 binding protein AEBP2 [M. musculus] {IMAG | AA416308.1 |
| 2.2 | 556 | 1207 | ESTs, Highly similar to Bdeight protein [R. norvegicus] {IMAGE:622724} | AA177555.1 |
| 2.5 | 426 | 1070 | ESTs, Highly similar to probable nocturnin protein [M. musculus] {IMAGE | AA051133.1 |
| 7.2 | 293 | 2099 | ESTs, Highly similar to purine nucleotide binding protein [M. musculus] { | AA240404.1 |
| 2.6 | 423 | 1108 | ESTs, Highly similar to spliceosomal protein SAP 155 [H. sapiens] {IMAG | AA200809.1 |
| 3.6 | 124 | 451 | ESTs, Moderately similar to ACTIN-LIKE PROTEIN 14D [Drosophila mela | AA451385.1 |
| 3.3 | 663 | 2170 | ESTs, Moderately similar to CHLORINE CHANNEL PROTEIN P64 [Bos ta | AA466508.1 |
| 2 | 197 | 391 | ESTs, Moderately similar to CHLORINE CHANNEL PROTEIN P64 [Bos ta | AA537234.1 |
| 2.4 | 188 | 442 | ESTs, Moderately similar to PLASMA-CELL MEMBRANE GLYCOPROTEI | AA016397.1 |
| 2.2 | 680 | 1508 | ESTs, Moderately similar to PRE-MRNA SPLICING FACTOR SRP20 [Hom | W83358.1 |
| 2.2 | 330 | 740 | ESTs, Weakly similar to CCAAT-BINDING TRANSCRIPTION FACTOR SUE | AA413894.1 |
| 2.9 | 785 | 2288 | ESTs, Weakly similar to LYMPHOCYTE ANTIGEN LY-6A.2/LY-6E.1 PREC | AA472994.1 |
| 2.5 | 160 | 403 | ESTs, Weakly similar to lysophospholipase I [M. musculus] {IMAGE:59647 | AA153793.1 |
| 2.8 | 1172 | 3251 | ESTs, Weakly similar to mannose-binding lectin associated serine prote | AA209006.1 |
| 2.8 | 625 | 1738 | ESTs, Weakly similar to SDP8 [M. musculus] {IMAGE:747180} | AA260397.1 |
| 2.1 | 413 | 871 | ESTs, Weakly similar to Trif [M. musculus] {IMAGE:523380} | AA080169.1 |
| 3.3 | 214 | 706 | expressed sequence tag mouse EST 25 {IMAGE:638284} | AA200747.1 |
| 2.1 | 713 | 1511 | interferon activated gene 203 {IMAGE:618572} | AA170251.1 |
| 2.7 | 255 | 697 | interferon activated gene 203 {IMAGE:620541} | AA174447.1 |
| 4.8 | 1249 | 5945 | interferon inducible protein 1 {IMAGE:651927} | AA214784.1 |
| 9.6 | 411 | 3937 | interferon regulatory factor 1 {IMAGE:777393} | AA276697.1 |
| 2.8 | 2057 | 5726 | lymphocyte antigen 6 complex {IMAGE:580715} | AA145865.1 |
| 2.5 | 792 | 1977 | lymphocyte antigen 6 complex, locus C {IMAGE:425855} | AA000712.1 |
| 4.4 | 225 | 980 | monokine induced by gamma interferon {IMAGE:874758} | AA472492.1 |
| 21 | 204 | 4277 | Mus musculus Interferon-induced guanylate binding protein GBP-2 (Gbp | AA153021.1 |
| 2.8 | 279 | 777 | Mus musculus mRNA for GTPI protein {IMAGE:621346} | AA178613.1 |
| 2 | 1359 | 2725 | Mus musculus mRNA for heterogeneous nuclear ribonucleoprotein H {IM | AA003085.1 |
| 4.4 | 125 | 549 | Mus musculus mRNA for IIGP protein {IMAGE:747208} | AA260490.1 |
| 5.6 | 229 | 1293 | Mus musculus mRNA for IIGP protein {IMAGE:764385} | AA277451.1 |
| 2 | 268 | 546 | myeloid differentiation primary response gene 88 {IMAGE:572317} | AA110412.1 |
| 4.8 | 228 | 1096 | nuclear factor of kappa light chain gene enhancer in B-cells Inhibitor, alp | AA204405.1 |
| 2 | 606 | 1236 | protein kinase C, delta {IMAGE:777583} | AA276844.1 |
| 3.6 | 184 | 664 | Public domain EST {IMAGE:482196} | AA059908.1 |
| 2.9 | 132 | 379 | Public domain EST {IMAGE:596231} | AA122443.1 |
| 5.8 | 105 | 614 | Public domain EST {IMAGE:597020} | AA138757.1 |
| 7.1 | 221 | 1571 | Public domain EST {IMAGE:617529} | AA174721.1 |
| 2 | 122 | 247 | Public domain EST {IMAGE:619686} | AA175973.1 |
| 3.4 | 138 | 471 | Public domain EST {IMAGE:653161} | AI390695.1 |
| 2 | 215 | 425 | Public domain EST {IMAGE:669831} | AA253750.1 |
| 2.3 | 411 | 935 | PublIc domain EST {IMAGE:733734} | AA272876.1 |
| 2.1 | 383 | 801 | Public domain EST {IMAGE:850178} | AA451403.1 |
| 2.7 | 1687 | 4542 | Public domain EST {IMAGE:874030} | AA472200.1 |
| 2.6 | 579 | 1531 | Purine-nucleoside phopshorylase {IMAGE:607469} | AA172867.1 |
| 7.8 | 222 | 1740 | signal transducer and activator of transcription 1 {IMAGE:749544} | AA388678.1 |
| 3 | 617 | 1833 | signal transducer and activator of transcription 3 {IMAGE:440571} | AA014643.1 |
| 3 | 666 | 1990 | small inducible cytokine subfamily D, 1 {IMAGE:761708} | AA272232.1 |
| 4.4 | 138 | 613 | transporter 1, ABC (ATP binding cassette) {IMAGE:576775} | AA123837.1 |
| 2.2 | 329 | 719 | tryptophanyl-tRNA synthetase {IMAGE:367765} | W53959.1 |
| 2 | 357 | 717 | Y box protein 3 {IMAGE:421546} | W97158.1 |
| 2.4 | 944 | 2275 | zinc finger protein 36 {IMAGE:641523} | AA200282.1 |

I claim:

1. A method for identifying an agent which modulates an TNF & IFN influenced cellular process or response, the method comprising:
a) exposing a sample of cells to TNF & IFN;
b) determining the level of expression in the sample of cells of one or more TNF & IFN stimulated genes (Tables 1 and 2) in the presence and absence of a selected agent; and
c) identifying that the agent modulates an TNF & IFN influenced cellular process or response when the expression of the one or more TNF & IFN stimulated genes in the cell sample in the presence of the agent differs from the expression of the one or more TNF & IFN stimulated genes in the absence of the agent.

2. A method for identifying an agent which modulates an TNF & IFN influenced cellular process or response, the method comprising:
a) exposing a sample of cells to TNF & IFN;
b) determining the activity in the sample of cells of the product of one or more TNF & IFN stimulated genes (Table 1 and 2) in the presence and absence of a selected agent; and
c) identifying that the agent modulates an TNF & IFN influenced cellular process or response when the activity of the product of the one or more TNF & IFN stimulated genes in the cell sample in the presence of the agent differs from the activity of the product of the one or more TNF & IFN stimulated genes in the absence of the agent.

3. A method for identifying an agent which modulates an TNF & IFN influenced cellular process or response, the method comprising:
a) providing a sample of cells;
b) determining the level of expression in the sample of cells of one or more TNF & IFN stimulated genes (Tables 1 and 2) in the presence and absence of a selected agent; and
c) identifying that the agent modulates an TNF & IFN influenced cellular process or response when the expression of the one or more TNF & IFN stimulated genes in the cell sample in the presence of the agent differs from the expression of the one or more TNF & IFN stimulated genes in the absence of the agent.

4. A method for identifying an agent which modulates an TNF & IFN influenced cellular process or response, the method comprising:
a) providing a sample of cells;
b) determining the activity in the sample of cells of the product of one or more TNF & IFN stimulated genes (Table 1 and 2) in the presence and absence of a selected agent; and
c) identifying that the agent modulates an TNF & IFN influenced cellular process or response when the activity of the product of the one or more TNF & IFN stimulated genes in the cell sample in the presence of the agent differs from the activity of the product of the one or more TNF & IFN stimulated genes in the absence of the agent.

5. A method for detecting or monitoring a cellular process or response that is influenced by TNF & IFN, the method comprising:
a) obtaining a sample of cells from a patient;
b) determining the level of expression in the sample of cells of one or more TNF & IFN stimulated genes (Tables 1 and 2); and
c) identifying that the cells in the sample of cells obtained from the patient are undergoing a cellular process or response that is influenced by TNF & IFN when the level of expression of the one or more TNF & IFN stimulated genes in the cell sample is increased relative to the level of expression of the one or more TNF & IFN stimulated genes in a control the sample.

6. A method for detecting or monitoring a cellular process or response that is influenced by TNF & IFN, the method comprising:
a) obtaining a sample of cells from a patient;
b) determining the level of activity in the sample of cells of the product of one or more TNF & IFN stimulated genes (Tables 1 and 2); and
c) identifying that the cells in the sample of cells obtained from the patient are undergoing a cellular process or response that is influenced by TNF & IFN when the level of activity of the product of the one or more TNF & IFN stimulated genes in the cell sample is increased relative to the activity of the product of the one or more TNF & IFN stimulated genes in a control the sample.

7. A method for assessing whether cells will be responsive to an agent which modulates an TNF & IFN influenced cellular process or response comprising the steps of
a) exposing a sample of cells obtained from a patient to a test agent;
b) determining the level of expression in the sample of cells of the one or more TNF & IFN stimulated genes (Tables 1 and 2) in the sample exposed to the agent and in a sample of cells that is not exposed to the agent; and
c) determining that the cells will be responsive to the agent when expression of the one or more of the TNF & IFN stimulated genes is altered in the presence of the agent.

8. A method for assessing whether cells will be responsive to an agent which modulates an TNF & IFN influenced cellular process or response comprising the steps of
a) exposing a sample of cells obtained from a patient to a test agent;
b) determining the level of activity of the product of the one or more TNF & IFN stimulated genes (Tables 1 and 2) in the sample of cells exposed to the agent and in a sample of cells that is not exposed to the agent; and
c) determining that the cells will be responsive to the agent when activity of the product of the one or more TNF & IFN stimulated genes in the cell sample in the presence of the agent differs from the activity of the product of the one or more INF & IFN stimulated genes in the absence of the agent.

9. A method for modulating an TNF & IFN influenced cellular process or response, the method comprising administering a compound which alters the expression or activity of an TNF & IFN stimulated gene (Tables 1 and 2).

10. The method of claim 10 wherein said compound is a TNF & IFN stimulated gene or the product thereof.

11. A method of treating a viral disease, disorder or infection, comprising the administration of a therapeutically effective dose of TNF & IFN which alters the expression or activity of an TNF & IFN stimulated gene (Tables 1 and 2).
